(19) United States
(12) Patent Application Publication
(10) Pub. No.: US 2005/0239193 A1
(43) Pub. Date: Oct. 27, 2005

(54) Device and method of use for detection and characterization of microorganisms and microparticles

(75) Inventors: Eric R. Henderson, Ames, IA (US); Saju R. Nettikadan, Ames, IA (US); Curtis L. Mosher, Ames, IA (US); James C. Johnson, St. Charles, IA (US)

Correspondence Address: MICHAEL BEST & FRIEDRICH, LLP, ONE SOUTH PINCKNEY STREET, P O BOX 1806, MADISON, WI, 53701, US

(73) Assignee: BioForce Nanosciences, Inc., Ames, IA (US)

(21) Appl. No.: 10/977,551

(22) Filed: Oct. 29, 2004

Related U.S. Application Data
(63) Continuation-in-part of application No. 10/160,372, filed on May 30, 2002, now Pat. No. 6,897,015.
(60) Provisional application No. 60/515,538, filed on Oct. 29, 2003.

(51) Int. Cl. C12Q 1/70; G06F 19/00; G01N 33/48; G01N 33/50; C12M 1/34

(52) U.S. Cl. 435/287.2; 702/19

(57) ABSTRACT

The present invention includes a method and apparatus for the detection of a microorganism or microparticle.

### CROSS-REFERENCE TO RELATED APPLICATION(S)

This application is a continuation in part of U.S. application Ser. No. 10/160,372, filed May 30, 2002 and claims benefit from U.S. Provisional Application Ser. No. 60/515,538 filed Oct. 29, 2003.

### FIELD OF THE INVENTION

The present invention relates to the detection and characterization of microorganisms, viruses, microparticles and other biological matter.

### BACKGROUND OF THE INVENTION

Microorganisms and microparticles can be difficult to detect because of their small size. This property, coupled with their ubiquitous nature, makes specific detection of a particular microorganism or microparticle challenging. Detection and identification of microorganisms or microparticles in relevant samples (e.g., water, air, blood, tissues, organs, etc.) are essential to minimize the transfer and spread of infections. Furthermore, quick identification of microorganisms may aid in devising effective treatment strategies. Detection and identification of microorganisms may also be useful in monitoring environmental contamination and in biodefense and biowarfare.

Microorganisms can infect agriculturally important plant and animal species, resulting in economic hardship. One class of microorganisms is viruses. Viral infections extol a great morbidity and mortality among the human population. Many of these infections result from undetected viruses in waters, foods and air and are promulgated by an ever-increasing interconnection of societies. Detection and identification of viruses in medically important samples such as blood, blood derivatives, tissues and organs can minimize the potential for transfer and spread of disease within hospitals and clinics.

Several popular methods for the detection and identification of viruses and other microorganisms exist. These methods generally fall into three categories: (a) Infectivity and infectivity reduction assays; (b) serology assays employing antibody detection to determine whether an individual has been exposed; and (c) direct assays in which antibodies are used to detect the presence of an antigen in the sample or nucleic acid-based assays in which elements of the microbial or viral genome are detected.

Infectivity-based assays are seldom used in diagnostics, yet both cell culture and animal-based amplification of virus in a sample may be necessary for many of the current diagnostic procedures. The use of animals in infectivity assays is costly, time consuming and subject to ethical dispute.

Serodiagnosis still exists in many-hospitals principally because there are no good alternatives for some infections. Serology is largely performed to determine antibody levels and to estimate the probability for infection. Antibody based tests are popular, but are usually limited to a battery of individual tests in a macroscopic format (e.g., Enzyme Linked ImmunoSorbant Assay, or ELISA).

Standard microbiological approaches to detect anthrax and other bacterial pathogens involve growth of the agent on nutrient agar and visual identification after various staining procedures. Carbon source utilization testing in various media identifies and differentiates among closely related isolates. Viral pathogens are usually identified after their administration, infection and amplification in animals, particularly embryonated eggs, mice or cell culture. This is the basic microbial identification scheme practiced today. While precise in their verification of pathogen identity, these procedures are very slow.

Nucleic acid based tests for detecting microorganisms typically rely upon the polymerase chain reaction (PCR). These tests can be subject to false positive and negative results and also only report on the presence or absence of nucleic acid fragments in a sample, which does not provide conclusive evidence for the presence or absence of a complete and infectious pathogen or microbe.

### BRIEF SUMMARY OF THE INVENTION

In one aspect, the present invention provides an apparatus or kit for detecting a microorganism or microparticle comprising a solid support comprising a surface adapted for use in a scanning probe microscope, a material deposited in a plurality of domains on the surface, wherein the material is capable of interacting with the microorganism or the microparticle, and wherein exposure of the apparatus to a sample which can contain the microorganism or the microparticle causes the microorganism or microparticle to interact with the material.

In another aspect, the invention provides for an apparatus for preserving a microorganism or microparticle comprising a solid support comprising a surface, a material deposited on the surface, wherein the material is capable of interacting with the microorganism or the microparticle, and wherein exposure of the apparatus to a sample which can contain the microorganism or the microparticle causes the microorganism or microparticle to interact with the material, and wherein the microorganism or microparticle is preserved while interacted with the material.

In another aspect, the invention provides for a method for detecting a microorganism or microparticle in a sample comprising, providing a substrate with a surface, depositing a material on the surface in a plurality of domains, wherein the material is capable of interacting with the microorganism or microparticle, exposing the material to the sample, and detecting an interaction between the material and the microorganism by imaging the interaction.

In another aspect, the invention provides a method for amplifying a nucleic acid within a microorganism comprising the steps of providing a substrate with a surface, depositing a material on the surface, wherein the material has a capacity to interact with the microorganism, exposing the material to the microorganism, wherein the microorganism comprises a nucleic acid, and wherein the microorganism interacts with the material, and amplifying the nucleic acid by exposing the microorganism interacted with the material to a polymerase chain reaction.

In another aspect, the invention provides a method for screening antibodies capable of capturing particulate antigens, comprising the steps of providing a substrate with a surface, depositing an antibody onto the surface, exposing the antibody to a component comprising a particulate antigen and measuring the interaction between the antibody and the particulate antigen using scanning force microscopy.

In another aspect, the invention provides a method of screening for an antiviral agent in a sample containing a virus and an antiviral agent comprising the steps of providing a substrate with a surface, depositing a capture reagent onto the surface, exposing the surface to the sample and detecting the virus that interacts with the capture reagent by atomic force microscopy.

In another aspect, the invention provides a method of screening for an antiviral agent comprising the steps of providing a probe suitable for use in atomic force microscopy, attaching a virus to the probe, contacting the probe with a surface, wherein the surface comprises a receptor that can interact with the virus, contacting the interaction between the receptor and the virus with an antiviral agent, and detecting a modulation in the interaction between the virus and the receptor.

In another aspect, the invention provides a method for detecting the presence of an antibody from an animal comprising the steps of providing a substrate with a surface, depositing the antibody on the surface, wherein the antibody is derived from the animal, and wherein the antibody is capable of interacting with a microorganism, exposing the antibody to the microorganism, and detecting an interaction between the material and the microorganism by imaging the material and the microorganism.

In another aspect, the invention provides an apparatus for detecting a microorganism or microparticle comprising a solid support comprising a surface adapted for use in diffraction assay, a material deposited in a plurality of linear arrays on the surface, wherein the material is capable of interacting with the microorganism or the microparticle, and wherein exposure of the apparatus to a sample which can contain the microorganism or the microparticle causes the microorganism or microparticle to interact with the material.

### BRIEF DESCRIPTION OF THE DRAWINGS

FIG. 1 is a graphical representation showing the specificity with which different coxsackie viruses were captured by a chip. The display scale optimizes the virus particle count for all samples, so that data for CB4, which was counted as approximately 2500 bound particles, is shown capped at the graph maximum of 500 particles.

FIG. 2a is a graphical representation showing the RT-PCR amplification of coxsackie virus B4 RNA from complex samples seeded with coxsackievirus B4.

FIG. 2b is a graphical representation showing the RT-PCR amplification of coxsackievirus B4 RNA from complex samples seeded with coxsackievirus B4 following immunocapture of the seeded sample using a chip.

### DETAILED DESCRIPTION

The invention described herein includes an affinity capture substrate that can be read by a variety of methods including, but not limited, to fluorescence, surface plasmon resonance, mass spectrometry, quartz crystal resonance, electron microscopy and scanning probe microscopy. A preferred method is scanning probe microscopy (SPM), in particular atomic force microscopy (AFM). Use of an AFM or another type of SPM creates a methodology for a simple rapid, sensitive and high throughput method for detection of microorganisms, pathogens, biological matter, viruses, or microparticles etc. (Moloney et al., 2002, Ultramicroscopy 91 pp. 275-279). This method can be applied to detect components in a sample, including whole viruses, viral like particles, viral proteins, prions and viral nucleic acids as well as to distinguish between strains of similar microorganisms and biomaterials. Additionally, fluorescence or other methods commonly practiced for detection of biological binding events can be employed when desired. In another embodiment, the affinity capture substrate can be used to detect the presence of a microorganism by exposure of the nucleic acid of a captured microorganism to a polymerase chain reaction. Where the microorganism is an RNA virus, or otherwise the microorganism or microparticle comprises RNA, a reverse transcriptase can be used to facilitate the polymerase chain reaction.

As used herein the term “microorganism” is used to mean any sort of viral, bacterial, fungal, microbial or other matter that can be detected using the teachings of the present invention. The term “microorganism” as used herein may include any microorganism, whether naturally occurring or genetically engineered. Microorganisms may include, for example, viruses, eukaryotic microbes, bacteria, fungi, nanobacteria and parasites. The bacteria suitable for detection using the invention can be smaller than 500 nm and can include Rickettsaie, Chlamydia and Mycoplasma. Viruses that can be detected can include both infectious and noninfectious varieties, those used in the development of vaccines, for biodefense and biowarfare, and for gene therapy.

The term “microparticle” is used to mean any sort of viral vector, virus like particle, prion, bacterial spore, parasite, fungi spore, pollen grains or other component that can be detected using the teachings of the present invention. The term “microparticle” as used herein can be naturally occurring or genetically engineered. It can include, though is not limited to, a replication incompetent adenovirus, an adenoassociated viral vector, a human papilloma viral like particle, a human papilloma viral like particle of the type 16, type 18, type 31 or type 33, or a pox vector.

The term “sample” is a substance that is being tested to determine whether a target substance is contained therein. These samples can be natural or man-made substances. Alternatively the sample may be a biologically produced product or an artificially made product. The samples may be a solution, gas, or other medium.

The general applicability of the disclosed detection methods has been demonstrated for a wide variety of microorganisms and microparticles including, but not limited to, adenovirus, vaccinia virus, coxsackie virus, enterovirus, human papillomavirus, echovirus 6, echovirus 9, echovirus 11, echovirus 30, herpes simplex virus, parvovirus, bacteriophage, fd phage, Ms2 phage, polio virus, and Marek's disease virus and numerous virus like particles.

The Parvoviridae family of viruses contains members such as canine parvovirus, and are single-stranded, linear DNA viruses. Parvoviral particles are icosahedral, 18-26 nm in diameter and consist of protein (50%) and DNA (50%). The virions are not enveloped. The nucleocapsid comprises three capsid proteins, VP1, VP2 and VP3. Infectious virions of CPV contain 60 protein subunits, predominantly VP2.

The Caliciviridae family comprises single-stranded, linear RNA viruses with isometric capsids that typically have 32 cup-shaped depressions. They are 35-39 nm in diameter with icosahedral symmetry. The Norwalk virus, which causes gastroenteritis, lacks typical calicivirus morphology, but has a buoyant density and a single capsid polypeptide typical of caliciviruses.

The Papoviridae family contains 2 genera of oncogenic DNA viruses: papillomaviruses and polyomaviruses. Both polyomavirus and papillomaviruses genomes are double stranded, circular DNA molecules and have capsids formed from 72 pentameric capsomers. Polyomaviruses virions are non-enveloped, about 45 nm diameter, icosahedral particles and infect a wide variety of vertebrates. Polyomaviruses have 3 capsid proteins, VP1, VP2 and VP3. Papillomaviruses are non-enveloped icosahedral particles, approximately 52-55 nm diameter. Papillomaviruses have 2 capsid proteins, 1 major (encoded by the L1 gene) and 1 minor (L2). Papillomaviruses infect birds and mammals usually causing a benign outgrowth of cells.

The Picornaviridae family comprises linear, single-stranded RNA viruses, with non-enveloped, isometric nucleocapsids. They are 27-30 nm in diameter with icosahedral symmetry, and 12 capsomers per nucleocapsid. The family includes enteroviruses (e.g. coxsackie viruses, echoviruses) and rhinoviruses. Enterovirus infections are common in humans. Coxsackie viruses are associated with meningitis, paralysis and myocarditis.

The Leviviridae family comprises linear, single-stranded RNA viruses. The capsid is round, exhibits icosahedral symmetry, is isometric, consists of 32 capsomers and has a diameter of about 26 nm. The family includes the enterobacteria phage MS2, a non-enveloped bacteria-infecting phage with a diameter of approximately 20 mm.

The Inoviridae family comprise circular, single-stranded DNA viruses. Virions have a simple construction and consist of a non-enveloped, elongated, rod-shaped capsid that exhibits icosahedral symmetry. The family includes the enterobacteria phage fd, a filamentous phage of approximately 10 nm in diameter and 900 nm in length, which can infect E. coli.

Vaccinia virus belongs to the poxvirus family and is among the largest viruses known. The vaccinia virus is an analog of smallpox, a designated biowarfare element. The vaccinia virus is enveloped; slightly pleomorphic; ovoid, or brick-shaped. The viral particles are 140-260 nm in diameter and around 220-450 m long. The viral particle is composed of an external coat containing lipid and tubular or globular protein structures enclosing one or two lateral bodies and a core, which contains the genome.

It is envisioned that any virus may be detected using the apparatus and methods described herein without changing the nature or scope of the invention. The detected virus may belong to any viral family, including, for example, Adenoviridae, “African swine fever-like viruses,” Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae, Corticoviridae, Cystoviridae, Deltavirus, Dianthovirus, Enamovirus, Filoviridae, Flaviviridae, Furovirus, Fuselloviridae, Geminiviridae, Hepadnaviridae, Herpesviridae, Hordeivirus, Hypoviridae, Idaeovirus, Inoviridae, Iridoviridae, Leviviridae, Lipothrixviridae, Luteovirus, Machlomovirus, Marafivirus, Microviridae, Myoviridae, Necrovirus, Nodaviridae, Orthomyxoviridae, Papovaviridae, Paramyxoviridae, Partitiviridae, Parvoviridae, Phycodnaviridae, Picornaviridae, Plasmaviridae, Podoviridae, Polydnaviridae, Potexvirus, Potyviridae, Poxyiridae, Prions, Reovirida, Retroviridae, Rhabdoviridae, Rhizidiovirus, Satellites, Sequiviridae, Siphoviridae, Sobemovirus, Tectiviridae, Tenuivirus, Tetraviridae, Tobamovirus, Tobravirus, Togaviridae, Tombusviridae, Totiviridae, Trichovirus, Tymovirus and Umbravirus.

In one embodiment, the material used to capture these microorganisms or microparticles is an antibody. The antibody that interacts with the microorganism or microparticle may be naturally occurring or an engineered protein. In an alternative embodiment, the material can be a nucleic acid aptamer or protein aptamer. In another embodiment, the microorganism or microparticle can also be captured directly onto another material deposited on the surface of the chip.

The term “material” is the material deposited on the chip for which the microorganism or microparticle has some affinity, such as a binding agent. The material can be deposited in a domain, otherwise known as an deposition or affinity domain. The material and the microorganism or microparticle may interact through any means including, for example, electrostatic or hydrophobic interactions, covalent binding or ionic attachment. The material may include, but is not limited to, antibodies, proteins, peptides, nucleic acids, peptide aptamers, nucleic acid aptamers, or cellular receptors.

The term “chip” as used in the present invention includes a substrate that has a surface. The surface may be integral with the solid support of the chip, or may comprise a substance deposited on to the solid support. The “chip” may or may not include the material deposited thereon. The chip is exposed to the microorganism or microparticle to detect the microorganism or microparticle.

The surface of the chip may include a chemically activated surface including, but not limited to, silane, alkanethiolate and polyethylene glycol chemistries, produce surfaces on a chip amenable to subsequent analysis. For analysis using a scanning probe microscope a smooth surface is preferred, for example, a surface having a roughness of less than 5 nm over at least about 25 square micrometers. The substrate on which the surface is created can comprise glass, silicon or mica, although any substance can be used that is amenable to the surface chemistries deposited thereon. Various chemistries have been routinely used by the inventors in the construction of the chip surface, including, but not limited to, glass, silicon, silanes, wherein such silanes can include aminosilanes, mercaptosilanes, or aldehyde silanes, chromium, gold, silver, platinum, tungsten, polyethylene glycol linker, calixcrown derivatives, and alkanethiolates such as those with N-hydroxy-succinimide ester, carboxylic acid, or hydrazine groups. A calixcrown derivative is a derivative of a macropolycyclic molecule in which calixarene and crown ether moieties are present in the same framework. Chemical approaches that create surfaces with roughness equal to or greater than the expected topographical features of viruses are less desirable, although they may be usable with the appropriate analytical tools, such as an appropriate statistical method and particle counting approach.

In the case of antibodies, protein A, G or A/G may be used on the chemically activated surfaces to facilitate antibody orientation and improve virus capture efficiency. The capture efficiency is defined as the ratio between the numbers of virus particles captured in any defined area to the number of applied viruses. The virus capture efficiency of the material adsorbed to the chromium/gold substrate may be used as the basal standard by which other chemistries may be compared. A non-specific blocking buffer can be applied to the chip prior to the deposition of antibodies to block unreacted succinimide groups. The use of such buffers are known in the art and may contain 50 mM Tris pH 9.0, 150 mM sodium chloride, 10 mM magnesium chloride, 3 mg/ml casein and 0.05% NP-40 detergent. An example of a suitable buffer is Viriblock buffer (Bioforce Nanosciences, IA). These buffers may also be used when applying a sample.

Typical viral diameters are such that a viral particle will interact with several antibodies on a chip. It is therefore not necessary that every deposited antibody molecule be biologically active or have the correct orientation.

Antibodies, aptamers, components that provide a smooth surface on the chip or components that orient the antibody or aptamer can be deposited on the surface of a chip in a plurality of domains. A variety of methods for depositing the material onto the surface of the chip are envisioned. These include pipetting, the use of a microjet inkjet, the use of standard microarraying methods such as microcontact printing using pin tools, acoustic levitation, for example using a Labcyte Echo 550 compound reformatter (Labcyte, CA) and polymeric stamping, for example using polydimethylsiloxane. These methods are useful in depositing the material in a plurality of discrete domains on the chip, so that a number of different interactions, each in its own specific domain may be interrogated in a single scan using a scanning probe microscope.

A “plurality of domains” is defined to mean more than one discrete domain of a substance that is deposited onto a solid support, or a material that is deposited onto the surface of a solid support. In one embodiment the solid support and the surface will form a chip. Different materials that specifically interact with different microorganisms or microparticles may be deposited in separate and discrete domains onto the surface.

The microjet inkjet is a device similar to the inkjet used in an inkjet printer. The microjet apparatus and method uses a microdroplet to create a plurality of domains of the antibody, aptamer or orienting component on the surface. In a present embodiment, a single microjet, with a 30 μm nozzle, and a MicroJet III controller (MicroFab Technologies, Inc., Texas) was used. The microjet was mounted on a custom-built computer controlled stage with translation along the X, Y and Z axes. Deposition domains of anti-viral antibodies on the order of few tens of microns were created in the protein G surface.

The microjet method offers the advantage of extensive field testing and previous utilization in commercial applications involving genome arrays. The microjet uses a piezoelectric pump for precise delivery of fluids in the nanoliter to picoliter range. The microjet can consistently make arrays of antibodies with spot sizes in the 30-80 μm diameter range separated by 20 μm. Using spot sizes of 50 μm and inter spot distance of 20 μm, production of a 2×2 array in a 120×120 μm area can be achieved. The AFM scan range is approximately 120 μm and therefore can interrogate all four spots in a single AFM scan field.

Subsequent to antibody deposition, the substrates can be re-hydrated by placing them in a high humidity environment for 30 minutes, such an environment may be greater than 50% more preferably about 90%. Exposing the chip to high humidity and re-hydrating the chip helps the antibody to bind to the protein G surface.

A chip comprising a material capable of interacting with a microorgaism is exposed to a sample and evaluated for molecular interaction events between the deposited material and a microorganism or microparticle capable of interacting with the deposited material.

Chips can be analyzed by a variety of methods including, but not limited to, fluorescence microscopy, fluorescent molecular probes, surface plasmon resonance, mass spectrometry, scanning electron microscopy, transmission electron microscopy, nearfield optical microscopy, nearfield molecular resonance imaging, scanning probe microscopy, atomic force microscopy, spectrometry, interferometry and other methods known to those in the art. It is envisioned that this invention can encompass using devices such as optical diffraction, resonating cantilevers and quartz crystal surfaces, whereby the detection of the binding of viruses occurs through static interactions, for example that can cause the bending of microcantilevers or through resonance-based analyses.

A preferred method is analysis by atomic force microscopy. A useful AFM for this purpose is the Dimension 3100 AFM manufactured by Veeco/Digital Instruments (Santa Barbara, Calif.). Chips may be imaged using contact or Tapping™ mode with silicon nitride or silicon Ultralevers (Veeco), respectively. Alternative methods and cantilever types are also useful in some cases. A cantilever and tip type, along with the scanning methodology is chosen to best obtain the desired data. For example, rapid contact mode scanning is ideal for quick readout and low image quality while slower tapping mode readout is good for high image quality and morphological analysis. Alternative modes including but not limited to phase, friction, compliability, adhesion, force mapping and others known to those in the art are also useful under certain circumstances and may be used as desired. The scan size will be determined based on the virus dimensions and the pixel density required for clear resolution of the morphological features of the virus when desired. Viruses on the arrays will be detected, quantitated by particle counting or change in surface volume measurements or any other relevant method, and correlated with the specificity of the material, such as the antibody, at the domain to which they are bound. The number of virus particles bound to a domain may be determined by manual counting or by using a commercially available particle counting software algorithm or by any other preferred method known to those in the art.

In an AFM, the interactions between a sharp, micron-scale probe and a sample are monitored and regulated as the probe scans over the sample. Extremely fine control of the motion of the AFM probe is achieved using piezoelectric crystals. Thus, the AFM is capable of about 2 nm lateral resolution and <1 nm vertical resolution. This level of resolution gives the AFM the ability to detect changes in topography in the Angstrom range. The ability of AFM to detect height changes on the order of 1 nm has been utilized for the detection of antibody-antigen interaction; AFM has also been used to image nucleic acids, proteins, viruses, bacteria, live cells and other biological matter. The AFM can be operated in solution and is capable of identifying molecular binding events in near-real time. For a typical AFM immunoassay, the change in height is on the order of 1-3 nanometers (nm), providing a change in signal of 100% (from 3 to 6 nanometers, for example). For viruses, this change is much greater, on the order of 30 to 300 nm, providing a signal to noise ratio of 10 to 100 fold.

The detection of viruses by AFM has a sensitivity of 10³ TCID50/μl, which is sufficient to detect many of the medically relevant enteroviral infections. Viruses have been detected using the methods and apparatus of this invention to levels as low as 10³ particles per μl. Higher levels of sensitivity may be achieved, for example, using different antibodies or aptamers, using different reagents and buffer compositions, adding energy during chip preparation, such as by shaking, centrifuging, vibrating, agitating or rotating, incubating for different time frames, incubating under different temperatures, or adding flow using microfluidic systems.

In an alternative embodiment of the invention, the detection of the microorganism can be facilitated via amplification of the nucleic acid contained within the microorganism using the polymerase chain reaction (PCR) or by using a reverse-transcriptase polymerase chain reaction (RT-PCR). PCR is a method well known in the art for amplifying a nucleotide sequence using a heat-stable polymerase and two short base primers. In RT-PCR, RNA sequences can be amplified indirectly by using a reverse transcriptase to copy the RNA into complementary DNA. The complementary DNA is then amplified by PCR. Sequencing or other analysis of the amplified nucleic acid are well known in the art and would facilitate the identification of the microorganism.

### EXAMPLES

### Example 1

Methods for Construction and Analysis of the Chips

Substrate Preparation

Substrates were prepared from polished silicon wafers cut into 7 mm or 4 mm squares. The squares were ultrasonically cleaned in water and in absolute ethanol (30 min each). The polished surface of each substrate was sputter coated with 5 nm of chromium and 10 nm of gold using a dual gun, ion beam sputterer operating at 4 mA and 7 KeV (IBC 2000, South Bay Technologies, CA). Target areas (600 μm diameter) were created using copper EM grids (Electron Microscopy Sciences) as masks during sputtering. Alternatively, a 600 μm gold domain was created using a nickel, single slot, electron microscopy grid (GA600-Ni, Electron Microscopy Sciences).

For some analyses, the substrates were further treated. The gold-coated, patterned substrates were removed from the sputterer and immediately immersed in a freshly prepared alkanethiolate solution to allow self-assembling monolayer (SAM) formation. Amine-reactive surfaces were created by incubating the patterened gold surfaces in 0.5 mM solutions of dithiobis-succinimidyl undecanoate (DSU) (Dojindo, Japan) in 1,4-dioxane (Sigma, MO) for 3 hours in sealed jars at room temperature. The substrates were rinsed in a 1,4-dioxane rinse and blown dry with dry argon. The target areas were covered with recombinant protein A/G (Pierce, IL) at 1 mg/ml in PBS (10 mM phosphate buffer, 137 mM NaCl and 2.7 mM KCl) and allowed to react for 60 min at room temperature. The substrates were immersed in Viriblock (BioForce Nanosciences, IA), a non-specific blocking reagent, for 30 min to block unreacted succinimide groups. Each chip was rinsed in deionized, 0.2 μm filtered water, blown dry with argon and stored at −20° C. until used.

(b) Deposition of Antibodies

For studies investigating the effects of pH on the deposition of antibodies, the anti-viral antibodies were diluted in the appropriate buffers and 1 μl was deposited over the gold slot on the surface of a chip. To achieve different pH values, 50 mM sodium phosphate was used for pH 6.2, 6.8 and 7.4 and 50 mM sodium bicarbonate was used for pH 8.2, 9.0 and 9.6. For antibody concentration studies, the antibodies were diluted to the appropriate concentrations in 50 mM sodium phosphate, pH 7.1 or 50 mM sodium bicarbonate, pH 9.0 depending on the particulate nature of the antibodies at that pH. The chips were incubated for 60 min in a humid environment to facilitate binding. Unbound antibody was removed by directing a stream of distilled water over the chip surface. The chips were dried in a stream of dry argon.

In other analyses, the virus chip was constructed by placing 1 μl of the anti-viral antibody at 0.5 mg/ml in PBS on the protein A/G domain of a substrate at room temperature. The substrates with the antibody droplet were incubated for 60 min on wet filter paper in a sealed Petri dish to facilitate antibody binding. Unbound antibody was removed by directing a stream of filtered, distilled water over the chip surface. It was not necessary to block the antibody-free protein A/G surface since these viruses did not bind to AG surfaces. The prepared chips were washed in filtered, deionized water and dried in a stream of dry argon and either used immediately or stored for up to 60 days at −20° C. Chips were constructed with 600 μm diameter antibody domains against a single virus type on each chip.

(c) Virus Preparation

Infectious virus stocks were obtained from the American Tissue Culture Collection. Phage fd (15669-B2) was plaque purified several times in host E. coli K12 and grown at 33° C. to titers of 10¹²-10¹³/ml by overnight culture on log phase E. coli K12 in tryptose phosphate broth (Difco). Uninfected bacteria and debris were removed from the lysed cultures by centrifugation at 10,000 rpm at 4° C. for 20 min in a Sorvall SS-34 rotor. The supernatant fraction containing the phage was saved at −20° C.

Coxsackie viruses coxsackievirus B1 (VR-1032) strain Conn-5, coxsackievirus B2 (VR-29) strain Ohio-1, coxsackievirus B3 (VR-30) strain Nancy, coxsackievirus B4 (VR-18) strain J.V.B.-Benschoten, coxsackievirus B5 (VR-185) strain Faulkner and coxsackievirus B6 (VR-1037) strain Schmitt 1-15-21 were used to make virus stocks. Virus stocks were prepared by infecting buffalo green monkey kidney (BGMK) cells in either T-flasks or Blake bottles at a multiplicity of infection of approximately 3 after they had reached approximately 80% of confluency. Cells were maintained in RPMI-1640 medium supplemented with 7.5% fetal bovine serum (Gibco, Inc. N.Y.) and antibiotics. Cell passaging was done at near confluency using a 0.25% trypsin-EDTA mixture (Gibco, Inc. N.Y.). Lysates were collected 24-36 h post-infection and viruses were separated from lysed cells and debris by centrifugation and filtration (5,000×g, 15 min, 4° C.; 0.2 μm polyethersulfone membranes (Corning Costar, NY). The viruses were collected by centrifugation (140,000×g, 3 h at 4° C.). The virus-containing pellets were dislodged from the tubes by soaking in TEN buffer (10 mM Tris HCl, pH 7.8, 2 mM EDTA, and 100 mM NaCl) for 12 h at 4° C. The pellets were resuspended in TEN buffer by mixing and brief sonication at low power using a bath-type sonicator. The resuspended virus preparation was centrifuged (14,000×g, 2 min, 4° C.) to remove residual debris and any large aggregates of virus and virus containing debris. Each virus preparation was divided into aliquots and stored at −80° C. Virus titers, usually 10¹⁰ to 10¹¹ TCID50/ml (tissue culture infectious dose/ml), were determined in triplicate by endpoint dilution method using BGMK host cells (Lewis et al., 1983, Can. J Microbiol. 29, pp. 1661 et seq.)

(d) AFM or Fluorescent Imaging

A Dimension 3100 from Veeco-Digital Instruments (Santa Barbara, Calif.) equipped with a “g” scanner was utilized for the large-scale topography measurements by AFM. The chips were imaged in Tapping™ mode using silicon ultralevers (Veeco Instruments) under ambient conditions. Nanoscope III version 4.43r8 software from Digital Instruments was used for data capture. Images were modified by flattening for data analysis. For some analyses scan size was set to 25 μm². The scan size was determined based on the virus dimensions and the pixel density of data capture. In some analyses, the maximal surface area of a single scan that would provide adequate morphological information of the viruses at the captured pixel density of 512×512 was 25 μm².

For fluorescent imaging, a Nikon inverted microscope, TE 2000U, equipped with 40× oil objective and Chroma technology filter sets for Cy2™ and Alexa 594™ dyes was used. An Orca-ER (Hamamatsu) cooled CCD digital camera with 1.3 Megapixel resolution was used to collect images. Metamorph (Universal Imaging Corporation) was used for image capture and analysis.

### Example 2

Specificity of Virus Capture by the Chip

This example, which is presented for illustrative purposes only, describes the use of the AFM immunoassay system for the rapid detection and identification of all six types (B1 to B6) of the group B coxsackieviruses. However, this invention has been successful in detecting in samples a variety of different viruses and virus like particles, including those of the families Papoviridae (e.g. human papilloma virus), Parvoviridae (e.g. canine parvovirus), Caliciviridae (e.g. Norwalk virus) and Picornaviridae (e.g. echoviruses), as well as bacteriophages such as fd and Ms2. In this example, the AFM immunoassay system was also used to capture coxsackievirus B3 from body fluids and environmental samples. Chips were prepared with gold, chromium, amine-activated alkanethiolate, and protein A/G layers as described in Example 1. Viruses were prepared as described in Example 1. In the standard assay, experimental samples and controls in the non-specific blocking reagent Viriblock (Bioforce Nanosciences, IA) were brought to room temperature and a small volume (1 μl) of each was applied onto the antibody-coated domains on the chip. The chip was incubated without mixing or agitation at room temperature in a humid environment for 60 min. At the end of the adsorption period, chip surfaces were rinsed with a 3-5 second stream of deionized, filtered water from a wash bottle and rapidly blown dry under a stream of argon. Chips were mounted on metal discs and imaged by AFM under ambient conditions. Each experiment was repeated at least 3 times and 5 fields of 25 μm² (a dataset) were imaged on each chip for each data point. Virus detection was defined as one or more virus particles observed in each image of a dataset. The limit of detection was defined as the concentration at which at least one virus particle was observed in every image of a dataset.

(a) Specificity of Virus Capture

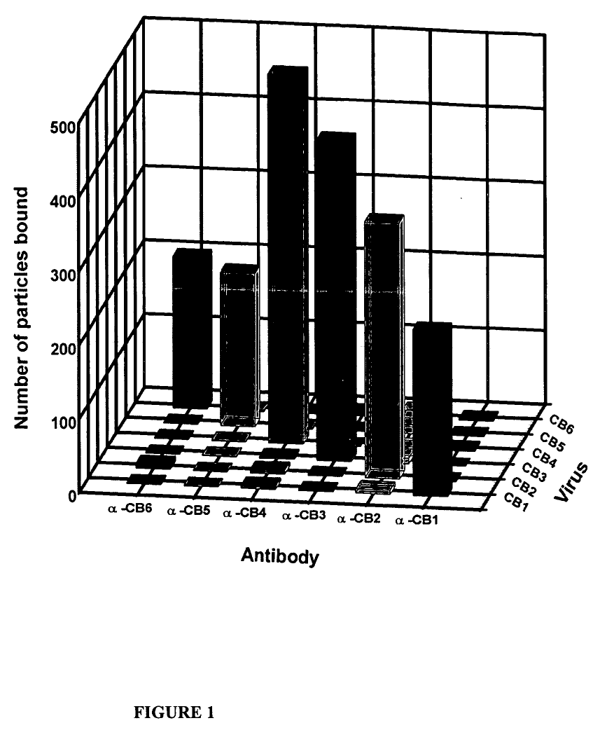

Chips were constructed with 600 μm diameter antibody domains against a single virus type on each chip. These chips were individually exposed to each of the six group B coxsackieviruses in the standard assay for this example. FIG. 1 shows a graphical representation of the mean bound virus count for each of the viruses and antibodies. The mean number of virus particles bound to a 25 μm² field in a 6×6 matrix of homologous antibodies or non-homologous antibodies on the chips was plotted. The display scale truncates the coxsackievirus B4 data, which had around 2500 particles bound, at the 500 particle maximum for the graph. As FIG. 1 shows, each of the 6 group B coxsackievirus types bound extensively to its specific chip with little or no binding observed on the non-specific chips. Under the conditions used in this example, up to 2500 particles bound to the specific homologous antibody surface while fewer than 10 particles were observed attached to the chips with non-homologous antibody. The viral particles could be readily identified by their distinct morphology and the uniformity of the particle shapes and sizes. Coxsackievirus particles could also be easily differentiated by this method from other particulates occasionally seen on surfaces. No virus binding was observed in the antibody-free regions of the chip.

Coxsackievirus B1, coxsackievirus B4 and coxsackievirus B6 were used to optimize the assay conditions and determine the limits of detection. In concentration dependent kinetic studies, 1 μl of each serial dilution of each these viruses was exposed to the corresponding specific chip. The binding kinetics as a function of concentration were linear, with a ten-fold increment in virus concentration resulting in a ten-fold increase in the number of virus particles captured within a range of 10³ TCID50 to 10⁷ TCID50 applied to the assay surface. At higher concentrations of virus, coverage of antibodies and spatial limitations on the capture surface limited the number of particles captured. The neutralization titer of the antibody correlated with the capture profile of the antibodies. The antibody against coxsackievirus B4 had a higher neutralization titer than anti-coxsackievirus B1 and anti-coxsackievirus B6. Also, saturation binding on anti-coxsackievirus B4 chips was reached at a lower virus concentration than was observed on anti-coxsackievirus B 1 chips or anti-coxsackievirus B6 chips. Under the conditions used, the AFM immunoassay for coxsackievirus B4 had a detection limit of 10³ TCID50 μl; the other viruses were detected at a minimum level of 10⁴ TCID50 μl.

(b) Specific Capture of Virus from Complex Samples

Grab samples of primary sludge were obtained from the Des Moines Wastewater treatment plant. Sputum and urine were obtained from human volunteers. Coxsackievirus B3 was inoculated into the crude samples (10⁸ TCID50/ml) and incubated at room temperature with continuous mixing for 30 min. Virus was separated from particulates and other components of samples as follows. Each sample and uninoculated control was adjusted to 100 mM Tris HCL, pH 7.5, 0.2 M NaCl, 5% (vol/vol) glycerol and 10% fetal bovine serum, mixed using a Vortex Jr. for 2 min, and centrifuged (10,000 g, 5 min, 4° C.). Samples of the supernatants were prepared in Viriblock (Bioforce Nanosciences, IA) and applied to a chip using the standard assay protocol.

Coxsackievirus B3 in urine, sputum and primary sludge were exposed to anti-coxsackievirus B3 chips as described the standard assay of this example. The chip captured the specific virus type from each of the different samples. In a single extraction protocol, 32%, 26% and 108% of the virus particles from primary sludge, sputum and urine, respectively, were detected relative to the control samples. The control samples contained coxsackievirus B3 in phosphate buffered saline. The level of contaminants and particulates in these samples did not significantly interfere with the capture of virus onto the surface. Very little debris was observed by AFM on these surfaces and such debris was easily distinguished from the virus particles.

(c) Elution of Infectious Virus from the Chip

Coxsackie virus B3 and B5 were captured on a chip prepared by direct printing of antibodies on protein A/G coated gold surfaces. Under these conditions the viruses were found to be inactivated while bound to the chip. However, the viruses could be eluted from the chip in glyine buffer. The eluted viruses were found to retain infectivity and could infect Buffalo Green Monkey Kidney Cells in cell culture.

### Example 3

Screening and Selection of Antibody Reagents

The efficiency of an antibody for capture of its particulate target can be evaluated by AFM imaging of the antibody domains and quantifying the number of particles captured (Nettikadan et al., 2003, Biochem Biophys Res Commun 311, pp. 540-5545). This process can be carried out rapidly in a multiplexed and automated fashion using existing instrumentation. It is feasible to screen a large number of antibodies, immobilized by various methods and on various surfaces, for their ability to bind specific particulate target species. In this example, the capture efficiencies of four anti-fd preparations adsorbed onto gold surfaces under various conditions, including pH and antibody concentration, were determined by AFM and compared.

Chips were constructed and analyzed according to example 1. In a standard assay, phage-containing samples and controls in Viriblock (BioForce Nanosciences, IA) were applied onto the antibody-coated region and the chip was incubated at room temperature in a humid environment for 30 min. The chip surface was rinsed with a stream of water, blown dry under a stream of argon and imaged by AFM using tapping mode. Each experiment was repeated at least 3 times and 5 fields of 50 μm² (a dataset) were imaged on each chip for each data point. Virus detection was defined as five or more virus particles observed in each and every image of a dataset. The limit of detection was defined as the concentration at which at least five virus particles were observed in every image of a dataset. The method described in this example offers a rapid, label-free and effective screening strategy to select capture reagents that interact specifically with microparticles or microorganisms. The method can be used to differentiate amongst surface immobilized antibodies based on the analysis of particle capture.

(a) Antibody Preparation

Anti-fd antibodies were purchased from four different sources. Rabbit anti-fd IgG (B 7786) was from Sigma-Aldrich (MO). This polyclonal antibody was noted to bind to fd coat proteins and would detect 5×10⁷ phage in an indirect SP-ELISA at a 1:1000 dilution using a peroxidase-labeled goat anti rabbit IgG. Mouse monoclonal anti-M13 IgG was purchased from Research Diagnostics, (NJ) (RDI-PRO61397). This antibody, a product of clone B62-FE2, was noted to have a detection limit of 10⁷ phage, at a working dilution of 1:5000 in ELISA. It bound to an internal epitope AEGDDPAKA and was noted for its tendency to precipitate at neutral pH. Mouse monoclonal anti-M13 IgG purchased from Amersham Pharmacia, (NJ) (27-9420-01) was a protein A column purified ascites suspension. Mouse monoclonal anti-M13 IgG purchased from Fitzgerald, (MA) (10-M24) was produced from clone M9909164, batch 739, using a M13 recombinant protein. This product was reported to be useful in immunohistochemistry and ELISA but no affinity constants were reported. Each antibody was received frozen supported by PBS at pH 7.2-7.4 with the exception of RDI-PRO61397 that was reconstituted with the addition of filtered, deionized water to produce a solution that was 0.02 M Tris-HCl at pH 8.0 with 60 mM NaCl. None of these preparations contained stabilizing proteins. The functionality and sensitivity of each of the antibodies was verified by immunoblot assay and by an ELISA using a kit from BIO-RAD (170-6432). Antibodies were deposited onto the chip as described in Example 1.

(b) Antibody Immobilization and Capture of Phage Fd

The optimum conditions of antibody immobilization that would generate the most particle-free, functional surfaces suitable for AFM was determined. AFM images of the four anti-fd antibody preparations individually adsorbed onto gold surfaces from a phosphate buffer at pH 7.2 were taken. The surfaces coated with antibody RDI-PRO61397 had extensive amounts of aggregates and was therefore not well suited to AFM analysis of phage capture. This antibody preparation was found to aggregate/precipitate when maintained at pHs less that 9.0. Even at pH 9.6, numerous aggregates were observed on the surface. The monoclonal antibody 10-M24 and the polyclonal antibody B7786 showed little particulate matter at pH 7.2. The monoclonal antibody 27-9420-01 resulted in surfaces with the least particulate matter.

Images of captured fd phage on the four antibody-coated surfaces were taken. Although the same amounts of antibody were used in the construction of these surfaces, and the surfaces were exposed to the same volume and concentration of fd, different numbers of fd particles were captured. The monoclonal antibody, 10-M24, was the least effective in capturing fd, while the polyclonal antibody, B7786, and the monoclonal antibody, 27-9420-01, captured similar numbers of fd particles.

The monoclonal antibody, RDI-PRO61397 coated surfaces were found to have large amounts of particulates that made AFM readout difficult. However, phage particles and the particles on the surface resulting from antibody adsorption could be differentiated because the phage particles had a mean length of 900 nm and mean diameter of 10 nm by AFM, whereas the particles on the antibody-coated surfaces were globular with diameters less than 10 nm. Compared to the other antibodies, a larger number of phage particles was bound to the RDI-PRO61397 coated surfaces using 10-fold less antibody. The increased numbers of phage observed on this antibody surface could be due to several factors including a higher affinity constant, a lower dissociation constant or trapping due to the particulate nature of the antibody.

(c) Effect of pH and Antibody Concentration on Virus Capture

A pH of around 7.3 was optimal for antibodies B7786, 27-9420-01 and 10-M24 as determined by the relative numbers of fd captured in the standard assay. The fourth antibody, RDI-PRO61397, resulted in surfaces with extensive aggregation so that AFM analysis of fd binding was rendered difficult. In general, fewer aggregates were seen on chip surfaces when antibodies were immobilized in buffers of a higher pH (9.0). The particulates that were found on the surfaces of chips with B7786, 27-9420-01 and 10-M24 did not interfere with AFM analysis for the virus. The greatest numbers of fd particles were captured when the antibodies were immobilized at pH 7.2-7.4. The numbers of fd phage captured decreased when the pH was either increased or decreased from the optimal pH. When the 27-9420-01 antibody was immobilized from bicarbonate buffer at pH 8.0, there was a significant decrease in number of fd captured when compared to immobilization in phosphate buffer adjusted to the same pH. The use of PBS (10 mM phosphate buffer, 137 mM sodium chloride, 15 mM potassium chloride, pH 7.4) also resulted in an approximately 2-fold reduction in capture of fd when compared to 50 mM phosphate buffer. These findings suggest that both the anion type and salt concentration affect phage capture.

The effect of concentration of antibody in the immobilization scheme was investigated by incubating varying concentrations of the antibodies at their corresponding optimum pH. The three antibodies, 27-9420-01, 10-M24 and B7786 were immobilized at pH 7.1, while the other antibody, RDI-PRO61397, was immobilized at pH 9.0 because of the extent of interfering particulates on the chip surfaces. Antibody concentration was varied up to about 1.1 mg/ml. There was extensive variability, from about 1 particle up to 100 particles per 50 μm², in the number of fd particles captured by the different antibodies at a given antibody concentration. The efficiency of capture, defined as the ratio between average number of fd particles bound to 50 μm² antibody domains and the concentration of the applied antibody, also varied greatly. The RDI-PRO61397 monoclonal antibody was approximately 15-fold more efficient in the capture of fd phage than was the 27-9420-01 monoclonal antibody. The third monoclonal antibody, 10-M24, did not capture any fd phage particles when immobilized at concentrations up to approximately 1 mg/ml and captured very poorly when applied to the surfaces at even higher concentrations. The capture efficiency of the polyclonal antibody, B7786, was approximately 2-30 fold less than that of the two functional monoclonal antibodies, respectively, when applied at concentrations ranging from 0.05 to 0.5 mg/ml. This finding is as expected since the polyclonal antibodies are raised against multiple epitopes, many of which may not be accessible to immobilized antibodies.

Increasing the applied antibody concentrations further was without benefit as the maximum number of fd particles captured was limited to approximately 160 due to the spatial limitations of the target surface, the dimensions of the phage, and the pixel resolution of the images captured by AFM. Despite the high efficiency of capture of the RDI-PRO61397 antibody, it was not a suitable antibody for analysis by AFM because of the extensive aggregation observed on the capture surfaces even at low concentrations.

(d) Sensitivity of Virus Capture

The sensitivity of virus capture was tested for each of the four antibodies after immobilization onto gold at their optimum pH and concentrations. Sensitivity was defined as the lowest concentration of the applied fd sample that resulted in the capture of at least 5 fd particles in each of the 5 random 50 μm² fields examined. Antibody 27-9420-01 was immobilized using an applied pH of 7.1 and concentration of 0.4 mg/ml; antibody B7786 at pH of 7.1 and concentration of 2.0 mg/ml; antibody RDI-PRO61397 pH 9.0 and concentration of 0.2 mg/ml; and 10-M24 at pH 7.1 and concentration of 1.0 mg/ml. The chips coated with the antibodies were exposed to a two-fold dilution series of fd from a stock concentration of 5×10⁶ pfu/μl in the standard assay described in this example. The three antibodies captured differing amounts of fd at higher concentrations (5×10⁶ pfu/μl) of the phage applied to the chips. However, at lower fd concentrations (1×10⁵ pfu/μl)), the differences in numbers of fd particles bound was not as significant. At the highest applied fd concentration, the polyclonal antibody, B7786, captured the most particles (120), followed by 27-9420-01 (90), and RDI-PRO61397 (50). 10-M24 did not capture any particles. The antibodies B7786, 27-9420-01 and RDI-PRO61397 were all found to have sensitivities of approximately 10⁵ pfu/μl in the standard assay format.

### Example 4

Enhancement of Polymerase Chain Reaction Using a Chip

(a) Chip enhancement of the sensitivity of RT-PCR

Chips were prepared with chromium, gold, amine-activated alkanethiolate, and protein A/G layers as described in Example 1. A monoclonal antibody to coxsackie virus coxsackievirus B4 (MAB94, Chemicon International Inc.) was bound to the protein A/G domain to create a chip that was specific for capturing coxsackievirus B4.

AFM images of chips with anti-coxsackievirus B4 domains showed that the antibody-coated surface was relatively free of particulate matter. These chips were exposed to 1 μl of coxsackievirus B4 at a concentration of 10⁶ TCID50/μl (50% tissue culture infectious dose/μl). More than 500 coxsackievirus B4 particles bound to the chip.

To test the efficacy of the chip to enhance RT-PCR, the coxsackievirus B4 primer pair was tested for specificity against all six coxsackieviruses. Primers were designed against coxsackievirus B4 (strain J.V.B.-Benschoten, X05690) and were targeted downstream of the 5′ LTR within the coding regions of the capsid proteins. The RT-PCR reaction (25 μl) was carried out using the Qiagen OneStep RT-PCR Kit and the RT-PCR reaction products (20 μl) were separated on 1% agarose. The coxsackievirus B4 primers amplified only coxsackievirus B4 suggesting that no cross priming or mispriming occurred under these assay conditions.

The primer set was found to have a sensitivity of approximately 1 TCID50/μl by RT-PCR, when exposed to serial 10-fold dilutions of coxsackievirus B4. The signal intensity appeared to be proportional to the virus concentration. The virus particles were also captured onto chips from a series of coxsackievirus B4 virus dilutions prepared in Viriblock (BioForce Nanosciences) to prevent non-specific binding. The coxsackievirus B4 virus particles bound to the chips were used as templates in an RT-PCR reaction and the lowest level of detection by chip immunocapture and RT-PCR was 10⁻⁴ TCID50/μl. Thus, without prior nucleic acid extraction, the chip increased the sensitivity of RT-PCR.

(b) Detection of Virus in Complex Seeded Samples by PCR

The efficiency of RT-PCR to detect virus from complex seeded samples either directly or by using a chip was analyzed. Endogenous inhibitors within these samples are known to interfere with polymerase amplification. (Nettikadan et al., 2003, Biochem. Biophys. Res. Commun. 311 pp. 540-545; Al-Soud et al., 2000, J. of Clin. Microbiol. 38 pp. 4463-4470). Urine (from volunteers), caffeinated coffee and serum (Invitrogen, Grand Island, N.Y.) were filtered using a 0.2 μm syringe filter. Sputum (from volunteers) was mixed with equal volume of PBS (10 mM phosphate buffer, 137 mM NaCl and 2.7 mM KCl) and filtered using a 0.2 μm syringe filter. Random samples of primary sludge from the Des Moines Wastewater Treatment Plant in Iowa, were allowed to settle and the liquid containing smaller particulates above the sediment was used for all the sludge studies. Ten-fold serial dilutions of coxsackieviruses in 0.1 M Tris HCl (pH 7.4) containing 0.1 μg/ml bovine serum albumin (Jackson ImmunoResearch Laboratories Inc., West Grove, Pa.) were prepared. The experimental samples were seeded with serial dilutions of coxsackievirus B4 to a final concentration ranging from 106-10′ TCID50/μl. The samples and the unseeded controls were incubated at room temperature for 1 hour. The samples were centrifuged (4° C. for 5 min at 10,000 g), the supernatant collected and used in direct RT-PCR amplification or chip capture of viral particles followed by RT-PCR amplification.

A small volume (1 μl) of the complex samples seeded with serial ten-fold dilutions of coxsackievirus B4 was either added directly to the RT-PCR reaction mix or incubated on a chip for 1 hour. The chip was washed and added to the RT-PCR reaction mix. Direct RT-PCR amplification was inhibited to varying levels by all of the complex samples. RT-PCR was completely inhibited by serum, sputum and coffee. Primary sludge extracts partially inhibited RT-PCR with a resultant sensitivity of only 10⁶ TCID₅₀/μl. Urine samples inhibited RT-PCR the least, with a resultant sensitivity of 10² TCID₅₀/μl.

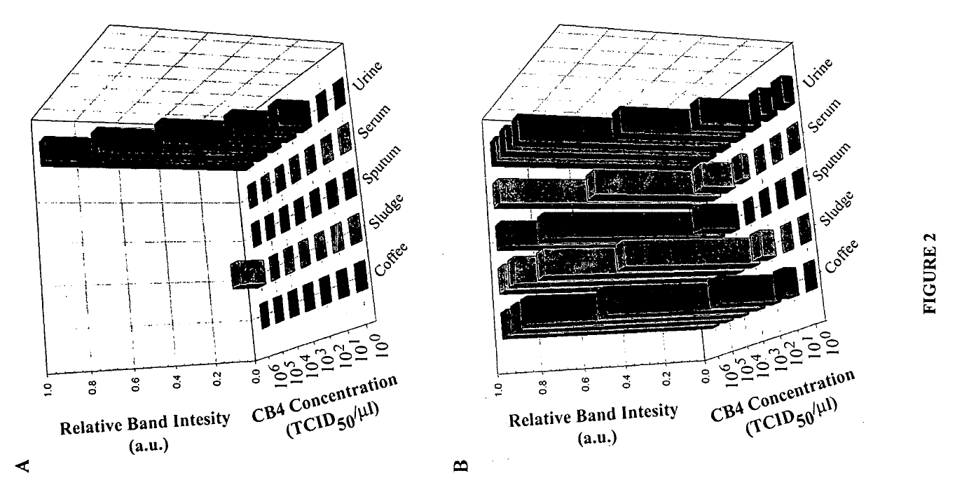

The capture of virus particles on the chip prior to RT-PCR facilitated the removal of some of the inhibitors present in the samples resulting in an increase in sensitivity. The maximum increase in sensitivity was obtained with coffee from complete inhibition to approximately 1 TCID₅₀/μl. Similarly, a 10², 10³ and 10⁵ TCID₅₀/μl increase in sensitivity was observed for coxsackievirus B4 inoculated urine, serum/sputum, and sludge, respectively. FIG. 2A is a graphical representation of the relative band intensity of PCR reactions performed using a 1 μl aliquot of the complex seeded samples. FIG. 2B is a graphical representation of the relative band intensity of PCR reactions performed using a chip that had been exposed to a 1 μl aliquot of the complex seeded samples. Virus immunocapture on a chip followed by RT-PCR amplification increased on average the sensitivity of RT-PCR by 10⁴ TCID50/μl.

Nucleic acid was extracted from coxsackievirus B4 diluted in Tris buffered saline, Viriblock, seeded sludge samples and chips incubated with samples of purified viruses and seeded sludge samples. Viral RNA was extracted using either a Qiagen kit, “QiaAmp Viral RNA Mini Kit,” or heat extraction by heating the virus sample for 5 min at 99° C. and immediately placing on ice. Similar RT-PCR sensitivity was achieved when using either heat extracted viral RNA or unextracted sample of coxsackievirus B4 diluted in Tris buffered saline as templates. When coxsackievirus B4 diluted in Viriblock or seeded sludge samples were used as templates, no amplification was observed in either the heat extracted or unextracted samples even at the highest concentration of virus used. However, using the chip to capture virus particles from Viriblock and seeded sludge samples resulted in a sensitivity of 10² TCID50/μl. Similar sensitivity was observed for both RNA extracted or unextracted samples from the chip. No noticeable increase in the RT-PCR sensitivity was observed between the extracted and non-extracted chip RT-PCR.

### Example 5

Use of the Chip as a Screening Device

This example describes the use of the chip in screening for antiviral agents. However, it is envisioned that this method can be used to screen for agents that are competitive or antagonistic to any microorganism or microparticle without changing the nature or scope of the invention.

(a) Competitive Inhibition of Virus Binding to the Chip

A chip was prepared using a specific capture reagent. In this example a coxsackievirus adenovirus receptor was used. A capture reagent can include other receptors such as antibodies or cellular receptors that are capable of capturing a desired virus type. The virus can be incubated on the chip in the presence and absence of a candidate competitor molecule. In this case the candidate competitor molecule was an antibody specific to coxsackievirus B3. However other candidate competitor molecules are envisioned, including, but not limited to, pharmaceuticals, cell surface receptors, chemical libraries, peptide libraries, nucleic acid libraries, exiting and new drug compositions, cell lysates, subcellular fractions, complex mixtures, blood, serum, and dissolved gases. The degree of binding of virus to the chip under these conditions was compared and correlated with the efficacy of the competitor molecular in terms of its ability to prevent virus-antibody binding. The degree of binding was measured using an atomic force microscope. Upon addition of the antibodies specific to coxsackievirus B3 in to the reaction mix, the capture of viruses onto the coxsackie adenovirus receptor surface was inhibited. Antibodies that were not specific to coxsackievirus B3 did not change the binding characteristics of the coxsackie adenovirus receptor surface. In some embodiments, this experiment can be carried out with cell surface proteins that bind to viruses as part of the infective process, wherein molecules that interrupt this binding interaction are good candidates for further analysis as anti-viral agents.

The multiplex nature of chip interface permits multiple virus-receptor interactions to be screened simultaneously. In addition, the exposure of the chip to competitor molecules may occur in a flow-through system, that permits washing and re-interrogation of the surfaces in the presence of additional potential competitor molecules.

(b) Competitive Inhibition of Virus Attached to Probe

Competitive inhibition of virus binding can also be detected using a direct force measurement method. (U.S. Pat. No. 5,372,930). A virus is directly attached to a force probe, such as an atomic force microscopy probe, and allowed to interact with receptor proteins on the chip surface. Attachment of the virus to the probe can be through a number of chemical approaches known to those in the field. These include, but are not limited to, succinimide-amine, thiol-gold, epoxide, carbodimide-carboxyl, silane and other biochemical approaches. The modified force probe is brought into contact with the chip and then pulled away. Binding between the virus on the probe and the receptor on the chip surface causes an interaction that results in bending of the force probe upon retraction from the surface. This force is measured as a function of the spring constant of the force probe. The same protocol is repeated upon addition of potential competitor molecules. When effective competitors are present, the binding force will be reduced. By evaluating the effectiveness of various candidate competitor molecules, candidates with possible anti-viral activity can be discovered. Integration of this system with a microfluidics system enables large numbers of molecules to be screened efficiently.

### Example 6

Use of the Chip to Detect Infection in an Animal

In many cases of acute infection of an animal, the titer of relevant antibodies in the serum will be sufficiently high to allow the construction of chips from relatively unpurified samples. In such a case, extracted serum may contain sufficient numbers of the relevant antibody species to allow production of chips from clear serum directly. Alternatively, if the titer of antibodies is not sufficiently high to allow the construction of chips from relatively unpurified samples, antibodies can be separated from an infected animal by preparing subcategories of antibodies using standard methods known in the art. For example, IgG may be separated from other classes of antibodies using affinity chromatography that is specific for the IgG sub-type using protein A or protein G column chromatography. Additional separation methods can include high performance liquid chromatography and electrophoresis.

In one embodiment, antibodies from an animal can be displayed on a chip and interrogated with known viruses to discover which specific viral antibodies the animal harbors. The chip could be exposed to a broad range of viruses or other microorganisms deposited in discrete domains onto the surface of the chip. Microorganisms of different genetic types could be exposed to the antibody in one chip.

Viruses or other microorganisms captured on the chip made using antibodies from the infected animal could be characterized by any of the methods described herein including, but not limited to, AFM, electron microscopy, scanning electron microscopy, cell culture, PCR, and immunodiagnostic methods. This rapid characterization of the virus could facilitate further analysis such as vaccine production.

In an alternative embodiment, a sample is taken from an infected animal or human wherein the infectious agent is not known. Antibodies from the infected animal or human are deposited on to a chip, and the chip is exposed to a sample taken from the same, or other, infected animal or human. The binding of a virus to the chip can be detected by methods including, but not limited to, a polymerase chain reaction, atomic force microscopy, mass spectrophotometry, electron microscopy, transmission electron microscopy, scanning electron microscopy, scanning probe microscopy, and high performance liquid chromatography. In this embodiment the virus that is detected may be of new type that had not been previously identified or known. This method can therefore facilitate the production of new vaccines and in the identification of viruses associated with diseases such as cancer.

A “genetic type” is defined to mean a strain or species that comprises a different genetic code than another different strain or species. Different genetic types can be different species or variants within a species that have different phenotypes or infectious properties.

### Example 7

Readout of the Chip by a Diffraction Mechanism

In this example, virus binding to a chip is detected by optical diffraction. A chip is prepared with capture domains deposited onto the surface of the chip as lines. In this embodiment, a preferred solid support comprises polystyrene plastic. The linear capture domains can be created by several methods known to those skilled in the art, such as ink-jet printing or direct microcontact printing. In this example, a chip for two different viral strains is prepared by microcontact printing a first antibody in linear arrays with line dimensions of about 1 μm wide by 2 mm long. A second antibody is similarly printed, but after rotation of the stamping tool by approximately 90 degrees to create an array of lines that intersect the first array. In other embodiments the capture domains may comprise aptamers, proteins or cell receptors.

A diffraction pattern is generated using a 10 mW laser (532 nm wavelength) that impinges upon the patterned surface and projects a diffraction image on a charged-coupled device detector. The chip is contacted with a sample that contains a virus capable of binding to the antibodies deposited on the chip. The binding event between the virus and the antibodies results in a change in the diffraction pattern intensity. This change in intensity can be used to detect the binding event. The portion of the diffraction pattern that changes indicates to those of ordinary skill in the art, which of the two antibody species on the surface has been bound by virus particles (Goh et al., 2002, Anal Bioanal Chem 374 pp. 54-56).

### Alternative Embodiments

In one alternative embodiment, larger spots can be used in conjunction with a mechanism for rapid translation of the sample under the scanning probe. In this embodiment, spot sizes of 60-100 μm diameter can be positioned a few tens of microns apart in the array. The AFM would then scan the spots in a known order, relying on accurate translation of the spots to the interrogation field. This operation may be readily accomplished using a conventional high-resolution translation stage. Throughput would not be compromised because faster scan rates (e.g., 3 Hz vs. 1 Hz) and lower resolution (e.g. 256 vs. 512 lines per scan) data could be employed to offset the additional time required to physically translate the stage without introducing intolerable degradation of data.

The information and examples described herein are for illustrative purposes and are not meant to exclude any derivations or alternative methods that are within the conceptual context of the invention. It is contemplated that various deviations can be made to this embodiment without deviating from the scope of the present invention. Accordingly, it is intended that the scope of the present invention be dictated by the appended claims rather than by the foregoing description of this embodiment.

All references cited in this application are hereby incorporated by reference in their entirety.

1. An apparatus for capturing a microorganism or microparticle comprising:
a solid support comprising a surface adapted for use in a scanning probe microscope;
a material deposited in a plurality of discrete domains on the surface, wherein the material is capable of interacting with the microorganism or the microparticle, and wherein exposure of the apparatus to a sample which can contain the microorganism or the microparticle causes the microorganism or microparticle to interact with the material.

2. The apparatus of claim 1, wherein the support comprises glass, silicon or mica.

3. The apparatus of claim 1, wherein the surface is deposited on the support in discrete regions.

4. The apparatus of claim 1, wherein the scanning probe microscope is an atomic force microscope.

5. The apparatus of claim 1, wherein the surface comprises a smooth plane.

6. The apparatus of claim 5, wherein the surface has a roughness of less than 5 nm over 25 square micrometers.

7. The apparatus of claim 1, wherein the substance comprises a moiety selected from the group consisting of gold, chromium, platinum, silver, a silane, a polyethylene glycol linker, a calixcrown derivative, silver, tungsten, silicon, glass or mica.

8. The apparatus of claim 1, wherein the material comprises a moiety selected from the group consisting of a silane, a polyethylene glycol linker, a calixcrown derivative, gold, chromium, platinum, silver, tungsten, an alkane ethiolate, an alkane linker, protein A, protein G, protein A/G, an antibody, or an aptamer.

9. The apparatus of claim 1, wherein the support and the surface form a chip.

10. The apparatus of claim 1, wherein the microorganism is a virus.

11. The apparatus of claim 10, wherein the virus is selected from the group consisting of an adenovirus, a vaccinia virus, a herpes simplex virus, a coxsackie virus, a human papilloma virus, an enterovirus, an echovirus 6, an echovirus 9, an echovirus 11, an echovirus 30, a parvovirus, a bacteriophage, a fd phage, a Ms2 phage, a polio virus, or a Marek's disease virus.

12. The apparatus of claim 1 comprising:
a solid support comprising a surface;
a material deposited on the surface, wherein the material is capable of interacting with the microorganism or the microparticle, and wherein exposure of the apparatus to a sample which can contain the microorganism or the microparticle causes the microorganism or microparticle to interact with the material and wherein the microorganism or microparticle is preserved while interacted with the material.

13. The apparatus of claim 12, wherein the microorganism is inactivated on the surface.

14. The apparatus of claim 13, wherein the microorganism can be eluted from the material and wherein the microorganism retains activity.

15. A method for detecting a microorganism or microparticle in a sample comprising:
providing a substrate with a surface;
depositing a material on the surface in a plurality of discrete domains, wherein the material is capable of interacting with the microorganism or microparticle;
exposing the material to the sample; and
detecting an interaction between the material and the microorganism by imaging the interaction.

16. The method of claim 15, wherein the surface further comprises a substance providing a smooth plane.

17. The method of claim 16, wherein the smooth plane has a roughness of less than 5 nm over 25 square micrometers.

18. The method of claim 16, wherein the substance comprises at least one of gold, chromium, platinum, a silane, a polyethylene glycol linker, a Calixcrown derivative, silver, tungsten, silicon, glass or mica.

19. The method of claim 16, wherein the material comprises a moiety selected from the group consisting of a silane, a polyethylene glycol linker, a calixcrown derivative, gold, chromium, platinum, silver, tungsten, or an alkane ethiolate.

20. The method of claim 15, wherein the interaction is imaged using a microscope.

21. The method of claim 20, wherein the microscope is a scanning probe microscope.

22. The method of claim 21, wherein the scanning probe microscope is an atomic force microscope.

23. The method of claim 20, wherein the microscope is an electron microscope.

24. The method of claim 15, wherein the interaction creates a change in fluorescence.

25. The method of claim 24, wherein the interaction is imaged by spectrophotometry.

26. The method of claim 15, wherein the material comprises a moiety selected from the group consisting of a silane, a polyethylene glycol linker, a calixcrown derivative, gold, chromium, platinum, silver, tungsten, an alkane ethiolate, protein A, protein G, protein A/G, an antibody, or an aptamer.

27. The method of claim 15, wherein the surface comprises glass, mica or silicon.

28. The method of claim 15, wherein the microorganism is a virus.

29. The method of claim 28, wherein the sample comprises an adenovirus, a vaccinia virus, a herpes simplex virus, a coxsackie virus, a human papilloma virus, an enterovirus, an echovirus 6, an echovirus 9, an echovirus 11, an echovirus 30, a parvovirus, a bacteriophage, a fd phage, a Ms2 phage, a polio virus, or a Marek's disease virus.

30. The method of claim 15, wherein the microorganism is less than 500 nm in diameter.

31. The method of claim 15 wherein the microparticle comprises a prion, a virus-like particle or a viral vector.

32. The method of claim 15, wherein more than one microorganism is detected simultaneously.

33. The method of claim 15, wherein the domains are deposited using a piezo electric device.

34. The method of claim 15, wherein the domains are deposited using a device selected from the group consisting of a pipette, a contact printer with a pin tool, and acoustic levitator

35. A method for amplifying a nucleic acid within a microorganism comprising:
(a) providing a substrate with a surface;
(b) depositing a material on the surface, wherein the material has a capacity to interact with the microorganism;
(c) exposing the material to a sample which can contain the microorganism, wherein the microorganism comprises a nucleic acid, and wherein the microorganism interacts with the material; and
(d) amplifying the nucleic acid of step c in a polymerase chain reaction.

36. The method of claim 35, wherein the nucleic acid is a ribonucleic acid, and wherein the ribonucleic acid is amplified by contacting the ribonucleic acid with a reverse transcriptase prior to the polymerase chain reaction.

37. The method of claim 36, wherein the microorganism is a virus.

38. The method of claim 35, wherein the sample comprises an inhibitor of the polymerase chain reaction assay.

39. The method of claim 38, wherein the inhibitor is selected from the group consisting of urine, coffee, caffeine, serum, sputum or wastewater sludge.

40. The method of claim 35, wherein the surface comprises a gold layer, and wherein the material comprises an amine activated alkanethiolate.

41. The method of claim 40, wherein the material further comprises an orienting layer and an antibody.

42. The method of claim 35, wherein the surface further comprises a substance providing a smooth plane.

43. The method of claim 42, wherein the substance comprises a moiety selected from the group consisting of gold, chromium, platinum, a silane, a polyethylene glycol linker, a Calixcrown derivative, silver, tungsten, silicon, glass or mica.

44. The method of claim 43, wherein the material comprises a moiety selected from the group consisting of a silane, a polyethylene glycol linker, a calixcrown derivative, gold, chromium, platinum, silver, tungsten, an alkane ethiolate, protein A, protein G, protein A/G, an antibody, or an aptamer.

45. The method of claim 35, wherein the material comprises a moiety selected from the group consisting of a silane, a polyethylene glycol linker, a calixcrown derivative, gold, chromium, platinum, silver, tungsten, an alkane ethiolate, protein A, protein G, protein A/G, an antibody, or an aptamer.

46. A kit for detecting a microorganism or microparticle comprising the apparatus of claim 1.

47. A method for screening antibodies capable of capturing particulate antigens comprising:
providing a substrate with a surface;
depositing an antibody onto the surface;
exposing the antibody to a component comprising a particulate antigen; and
measuring the interaction between the antibody and the particulate antigen using scanning force microscopy.

48. The method of claim 47, wherein the scanning force microscopy comprises atomic force microscopy.

49. A method of screening for an antiviral agent comprising:
providing a substrate with a surface;
depositing a capture reagent onto the surface;
exposing the surface to sample which can contain a virus and an antiviral agent; and
detecting the virus that interacts with the capture reagent using scanning probe microscopy.

50. A method of screening for an antiviral agent comprising:
(a) providing a probe suitable for use in atomic force microscopy;
(b) attaching a virus to the probe;
(c) contacting the probe with a surface, wherein the surface comprises a material that can interact with the virus;
(d) contacting the receptor and the virus of step (c) with a sample that can contain an antiviral agent; and
(e) detecting a change in the interaction between the virus and the receptor, wherein the change is correlated with the presence of an antiviral agent.

51. A method for detecting the presence of an antibody from an animal comprising:
providing a substrate with a surface;
depositing the antibody on the surface, wherein the antibody is derived from the animal, and wherein the antibody is capable of interacting with a microorganism; exposing the antibody to the microorganism; and
detecting an interaction between the antibody and the microorganism.

52. The method of claim 51, wherein the microorganism is deposited onto the antibody in a plurality of domains.

53. The method of claim 52 wherein the microorganism comprises a first genetic type and a second genetic type, and wherein the first genetic types is deposited in a first domain and wherein the second genetic type is deposited in a second domain.

54. A method for detecting the presence of a microorganism from an animal comprising:
providing a substrate with a surface;
depositing an antibody on the surface, wherein the antibody is derived from the animal, and wherein the antibody is capable of interacting with the microorganism;
exposing the antibody to the microorganism; and
detecting an interaction between the antibody and the microorganism.

55. The method of claim 54, wherein the microorganism is a virus.

56. An apparatus for detecting a microorganism or microparticle comprising:
a solid support comprising a surface adapted for use in diffraction assay;
a material deposited in a plurality of linear arrays on the surface, wherein the material is capable of interacting with the microorganism or the microparticle, and wherein exposure of the apparatus to a sample which can contain the microorganism or the microparticle causes the microorganism or microparticle to interact with the material.

57. The method of claim 56 wherein the surface comprises polystyrene plastic.
